(19) United States
(12) Patent Application Publication
(10) Pub. No.: US 2016/0157872 A1
(43) Pub. Date: Jun. 9, 2016

(54) Lumen Re-Entry System And Method

(71) Applicant: Cook Medical Technologies LLC, Bloomington, IN (US)

(72) Inventors: Logan Michael Cage, Bloomington, IN (US); David Christian Lentz, Bloomington, IN (US); Jeremy Schaeffer, Bloomington, IN (US)

(21) Appl. No.: 15/044,394

(22) Filed: Feb. 16, 2016

Related U.S. Application Data
(63) Continuation of application No. 14/085,345, filed on Nov. 20, 2013.
(60) Provisional application No. 61/728,861, filed on Nov. 21, 2012.

(51) Int. Cl. A61B 17/22; A61M 25/01

(52) U.S. Cl. CPC A61B 17/22; A61M 25/0108; A61M 25/0194; A61B 2017/22095; A61M 2025/0197

(57) ABSTRACT

A lumen re-entry system includes a catheter having an elongate tubular body defining a wire lumen extending from an open proximal end of the elongate tubular body to a distal opening through the elongate tubular body. An abrasion resistant tubular liner is positioned within the wire lumen and has a fixed position relative to the elongate tubular body. The abrasion resistant tubular liner defines a reduced wire lumen extending from an open proximal end of the abrasion resistant tubular liner to an open distal end of the abrasion resistant tubular liner. A puncture wire is configured for axial movement through the reduced wire lumen and has an angled distal segment terminating in a puncture tip. The angled distal segment is oriented at an angle between about 10 degrees to about 90 degrees relative to a central longitudinal axis of the puncture wire.

### CROSS REFERENCE TO RELATED APPLICATION

This Application claims the benefit of the filing date of U.S. Provisional Patent Application Ser. No. 61/728,861, filed Nov. 21, 2012.

### TECHNICAL FIELD

The present disclosure relates generally to a lumen re-entry system, and more particularly to a lumen re-entry system including a catheter having an abrasion resistant tubular liner positioned within a wire lumen of the catheter and a puncture wire having an angled distal segment terminating in a puncture tip.

### BACKGROUND

Thrombosis is the formation of a thrombus, or blood clot, within the vascular system of a patient. A blood clot typically occurs when blood hardens from a liquid to a solid. When attached to vessel walls, blood clots, and other substances, such as plaque or fat, may reduce or block blood flow downstream from the clot. Chronic total occlusion (CTO) is a complete blockage within the vascular system or, more particularly, within an arterial vessel, that obstructs blood flow. This blocked blood flow may prevent critical blood flow and oxygen from reaching certain tissues and, thus, may result in damage to the tissues. Regardless of the particular location of the clot within the vascular system, a clot or, in particular, a CTO, if left untreated, may cause serious damage and, in some cases, may become life threatening.

A wide variety of techniques are available for treating a CTO. For example, some percutaneous techniques include the use of pharmacological agents, also referred to as thrombolytic agents, to help dissolve the clots. Other percutaneous techniques may include the use of a wire guide and/or catheter to cross the occlusion and recanalize the vessel. However, crossing a CTO using a wire guide and/or catheter may be difficult and, oftentimes, impossible, due to the hardness of the clot or occlusion. During these recanalization procedures, it is common for the wire guide to be inadvertently advanced into the subintimal space of the vessel wall. Once the wire guide has entered the subintimal space, either inadvertently or intentionally, it may be possible to create a new lumen through the subintimal space that bypasses the clot, such as by performing an angioplasty procedure. However, it is often difficult to redirect the wire guide back into the true lumen of the vessel at a distal location relative to the occlusion.

An exemplary lumen re-entry device is described in U.S. Patent Application Publication No. 2007/0219464 to Davis et al. Specifically, the Davis et al. reference teaches a steerable guide wire having a sharpened re-entry tip. The guide wire comprises a hypotube having a helical coil attached to and extending from a distal end of the hypotube. A retaining ribbon is connected to the distal end of the hyopotube and is also connected to the sharpened re-entry tip. A deflection member is slidably disposed within the hypotube and has a distal end connected to the sharpened re-entry tip such that distal movement of the deflection member deflects the sharpened re-entry tip in one direction, while proximal movement of the deflection member deflects the sharpened re-entry tip in an opposite direction. While the lumen re-entry device of Davis et al. might offer successful deflection of the guide wire tip, the sharpened re-entry tip, which may be used for crossing an occlusion and/or re-entering a vessel lumen, may present risks of inadvertently puncturing or tearing the vessel wall during advancement and/or deflection.

The present disclosure is directed toward one or more of the problems set forth above.

### SUMMARY OF THE DISCLOSURE

In one aspect, a lumen re-entry system includes a catheter having an elongate tubular body defining a wire lumen extending from an open proximal end of the elongate tubular body to a distal opening through the elongate tubular body. An abrasion resistant tubular liner is positioned within the wire lumen and has a fixed position relative to the elongate tubular body. The abrasion resistant tubular liner defines a reduced wire lumen extending from an open proximal end of the abrasion resistant tubular liner to an open distal end of the abrasion resistant tubular liner. A puncture wire is configured for axial movement through the reduced wire lumen and has an angled distal segment terminating in a puncture tip. The angled distal segment is oriented at an angle between about 10 degrees to about 90 degrees relative to a central longitudinal axis of the puncture wire.

In another aspect, a lumen re-entry system includes a catheter having an elongate tubular body defining a wire lumen, and an abrasion resistant tubular liner affixed to the elongate tubular body within the wire lumen. The liner defines a reduced wire lumen extending from an open proximal liner end to an open distal liner end. The system further includes a puncture wire having a proximal segment defining a longitudinal axis, and an angled distal segment terminating in a puncture tip and oriented between about 10 degrees to about 90 degrees relative to the longitudinal axis. The puncture wire is movable from a first position within the reduced wire lumen where the puncture tip contacts the abrasion resistant tubular liner between the proximal and distal liner ends, to a second position where the puncture tip is advanced out of the lumen through the open distal liner end, for penetrating a vessel wall in a patient.

In still another aspect, a method of re-entering a lumen of a patient vessel using the lumen re-entry system includes advancing the catheter through a wall of the patient vessel and axially advancing the puncture wire through the reduced wire lumen with the angled distal segment oriented at an angle between about 10 degrees to about 90 degrees relative to a central longitudinal axis of the puncture wire. The abrasion resistant tubular liner is contacted with the puncture tip of the puncture wire during the axially advancing step. The puncture tip of the puncture wire is oriented such that the puncture tip faces the lumen of the patient vessel, and the lumen is entered with the puncture wire by axially advancing the puncture wire further through the reduced wire lumen such that the angled distal segment is advanced through the distal opening and the puncture tip penetrates the wall.

### BRIEF DESCRIPTION OF THE DRAWINGS

FIG. 1 is a partially sectioned side diagrammatic view of a lumen re-entry system, according to one embodiment of the present disclosure;

FIG. 2 is a sectioned side diagrammatic view of the lumen re-entry system catheter of FIG. 1, according to another embodiment of the present disclosure;

FIG. 3 is a perspective view of an alternative abrasion resistant tubular liner for the lumen re-entry system of FIG. 1;

FIG. 4 is a perspective view of another alternative abrasion resistant tubular liner for the lumen re-entry system of FIG. 1;

FIG. 5 is a side diagrammatic view of a vascular structure of a patient at one stage of a lumen re-entry procedure using the lumen re-entry system of FIG. 1;

FIG. 6 is a side diagrammatic view of the vascular structure at another stage of a lumen re-entry procedure using the lumen re-entry system of FIG. 1;

FIG. 7 is a side diagrammatic view of the vascular structure at another stage of a lumen re-entry procedure using the lumen re-entry system of FIG. 1;

FIG. 8 is a side diagrammatic view of the vascular structure at one stage of a vessel wall entry procedure using the lumen re-entry system of FIG. 1;

FIG. 9 is a side diagrammatic view of a lumen re-entry system according to another embodiment;

FIG. 10 is a sectioned side diagrammatic view of a part of the system of FIG. 9; and

FIG. 11 is a sectioned view taken along line 11-11 of FIG. 10.

### DETAILED DESCRIPTION

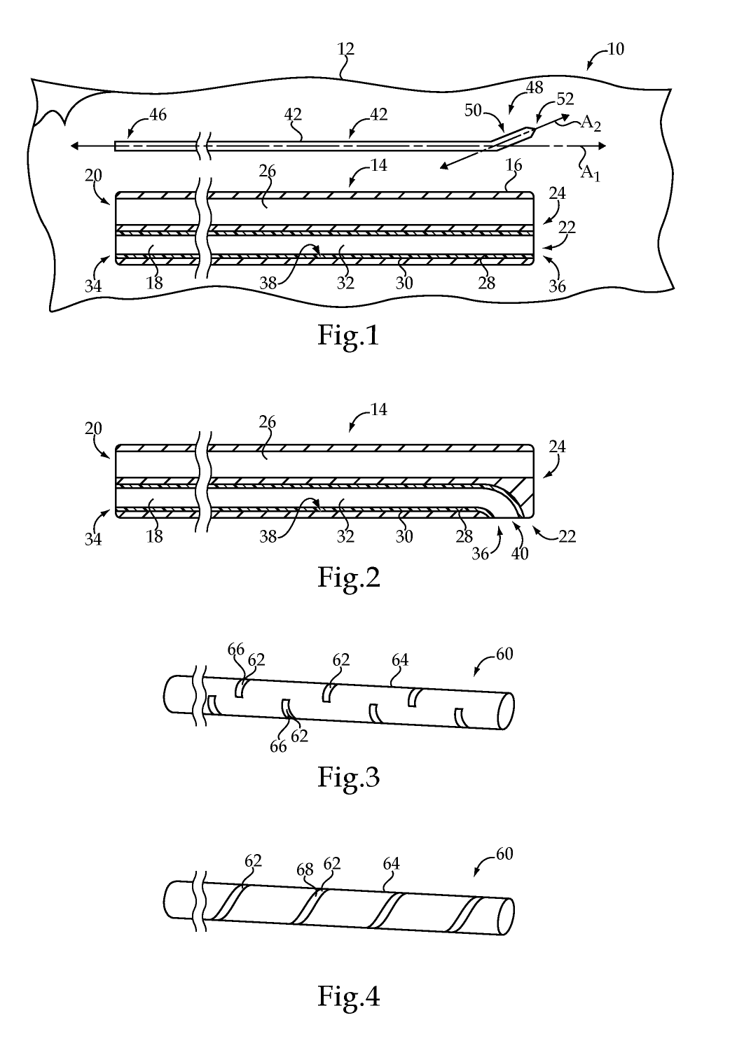

Referring to FIG. 1, there is shown a lumen re-entry system 10 according to one embodiment of the present disclosure. The lumen re-entry system 10 may include a number of components, which may be provided within a sterile, tear open package 12, as is known in the art. In performing a lumen re-entry procedure on a patient, some or all of the components of the lumen re-entry system 10 may be used, depending upon the specifics of the procedure to be performed. As should be appreciated, however, the components shown in FIG. 1 might be separately packaged and/or the lumen re-entry system 10 might also include components in addition to those shown, including components routinely used in percutaneous vascular procedures.

The lumen re-entry system 10 generally includes a catheter 14 having an elongate tubular body 16 defining a wire lumen 18 extending from an open proximal end 20 to a distal opening 22. As shown in FIG. 1, the wire lumen 18 may extend from the open proximal end 20 to an open distal end 24 of the elongate tubular body 16. In the present disclosure, “proximal” will be used to refer to the end of a component or feature that is closest to a clinician, while “distal” is used to refer to a component or feature that is farthest away from the clinician. Such meanings are consistent with conventional use of the terms and, as such, should be understood by those skilled in the art.

The elongate tubular body 16 may range in length from several inches to several feet long, and may have a catheter wall outer diameter that is orders of magnitude smaller than its length. The elongate tubular body 16 may be made from a common medical tube material, such as, for example, a plastic, rubber, or other polymer, such that the catheter 14 exhibits both stiffness, or firmness, and flexibility. The catheter 14 may include any of a variety of known configurations. According to some examples, the catheter 14 may include a tapered distal segment and/or may include a lubricious coating to facilitate movement of the catheter 14 through the vasculature of a patient.

As shown, the catheter 14 may be a dual lumen catheter. In particular, the elongate tubular body 16 may also define a working lumen 26, separate from the wire lumen 18, extending from the open proximal end 20 to the open distal end 24 in parallel with the wire lumen 18. However, some alternative embodiments may include a single lumen catheter, while other alternative embodiments may include various multiple lumen catheters. As will be described below, the wire lumen 18 may be configured to telescopically receive a wire guide, while the working lumen 26 may be configured to telescopically receive a variety of other medical devices commonly used in percutaneous procedures, and provides a conduit for the injection of fluid.

An abrasion resistant tubular liner 28 is positioned within the wire lumen 18 and has a fixed position relative to the body 16. The abrasion resistant tubular liner 28 generally includes a tubular body 30 defining a reduced wire lumen 32 of the catheter 14 and extending from an open proximal end 34 of the abrasion resistant tubular liner 28 to an open distal end 36 of the abrasion resistant tubular liner 28. The abrasion resistant tubular liner 28 may be adhered, melted, or otherwise affixed to a wall 38 defining the wire lumen 18, and may extend partially or entirely along the length of the catheter 14 and may have any desired thickness suitable for the purposes described herein. The abrasion resistant tubular liner 28 may also be made from a common medical tube material, such as a polymer, or, according to some embodiments, may be made from stainless steel or nitinol. Preferably the abrasion resistant tubular liner 28 is harder than, and will typically have a higher durometer than, the elongate tubular body 16 of the catheter 14. A polymer from which liner 28 is formed may be a different polymer than that from which body 16 is formed.

Although various alternative catheter configurations exist, one alternative embodiment is shown in FIG. 2. As shown, the wire lumen 18 may extend from the open proximal end 20 to a side port 40 through the elongate tubular body 16. The side port 40 may be proximally spaced from the open distal end 24 of the elongate tubular body 16. It should be appreciated that the wire lumen 18 may be in fluid communication with one or both of the side port 40 and the open distal end 24, depending on the particular application. Further, if applicable, the wire lumen 18 may include a curved segment or orthogonal segment terminating at the side port 40.

Returning to FIG. 1, the lumen re-entry system 10 also includes a puncture wire 42 configured for axial movement through the reduced wire lumen 32, which is defined by the abrasion resistant tubular liner 28 positioned within the wire lumen 18 of the catheter 14. Generally speaking, the puncture wire 42 may be similar to a conventional wire guide and, thus, may include an elongate flexible body 44 extending from a proximal end 46 to a distal end 48. However, the puncture wire 42 also includes an angled distal segment 50 terminating in a puncture tip 52. Preferably, the angled distal segment 50 is oriented at an angle between about 10 degrees to about 90 degrees relative to a central longitudinal axis A₁ of the puncture wire 42. According to some embodiments, the preferred angle of the angled distal segment 50 relative to the central longitudinal axis A₁ may be about 45 degrees. The angled distal segment 50 may be a preformed angled tip or may be formed by a clinician during a lumen re-entry procedure, as will be described below. The wire 42 may be movable from a first position within reduced wire lumen 32, at which the tip 52 contacts the liner 28 between ends 20 and 24, to a second, advanced position where the tip 52 is advanced out of the lumen 32 through end 24.

According to some embodiments, the puncture wire 42 may be made from a metallic material, such as stainless steel, or, alternatively, may be made from a common medical tube material, such as those described above with respect to the catheter 14 and abrasion resistant tubular liner 28. It is also desirable for the puncture wire 42 to exhibit both stiffness, or firmness, and flexibility. For example, the puncture wire 42 should be flexible enough to navigate through the reduced wire lumen 32 defined by the abrasion resistant tubular liner 28, but stiff enough to provide sufficient force for puncturing through a vasculature wall using the distal puncture tip 52.

The puncture wire 42 may include any of a variety of known configurations. For example, the puncture wire 42 may include an elongate core element with one or more tapered sections near a distal end thereof. According to all embodiments, however, the elongate flexible body 44 includes a relatively stiff angled distal segment 50 terminating in the distal puncture tip 52, which is configured to puncture through a vessel wall. Specifically, the distal puncture tip 52 may include a sharp needlepoint that points generally along an angled axis A₂ of the angled distal segment 50. The puncture wire 42 may also include a coating, such as a lubricious polymer coating, to facilitate movement of the puncture wire 42 within the catheter 14. The puncture wire 42 may preferably be longer in length than the catheter 14 to facilitate manipulation of the proximal end 46 of the puncture wire 42 by a clinician.

Turning now to FIG. 3, an exemplary embodiment of an abrasion resistant tubular liner 60 for use with the lumen re-entry system 10 is shown. Since the abrasion resistant tubular liner 60, when positioned as shown in FIG. 1, will increase the thickness of the catheter 14 at the wire lumen 18, one or more flexibility increasing cuts 62 may be provided through a wall 64 of the abrasion resistant tubular liner 60. The flexibility increasing cut, or cuts, 62 may be provided entirely through the wall 64 of the abrasion resistant tubular liner 60 or only partially through the wall 64 (i.e., may have a depth less than the wall thickness), and may have any width, or shape. As shown in FIG. 3, a plurality of discontinuous cuts 66 may be provided. Alternatively, as shown in FIG. 4, the at least one flexibility increasing cut 62 may include a single continuous spiral cut 68 through the liner wall 64. As previously stated, any number, shape, size, and pattern of cuts 62 may be provided through the wall 64 of the abrasion resistant tubular liner 60. The cuts 62 may be provided along the entire length of the abrasion resistant tubular liner 60 or may be provided along specific portions. It should be appreciated that such determinations may be made based on the desired flexibility required for the particular procedure or based on the ease of manufacturing.

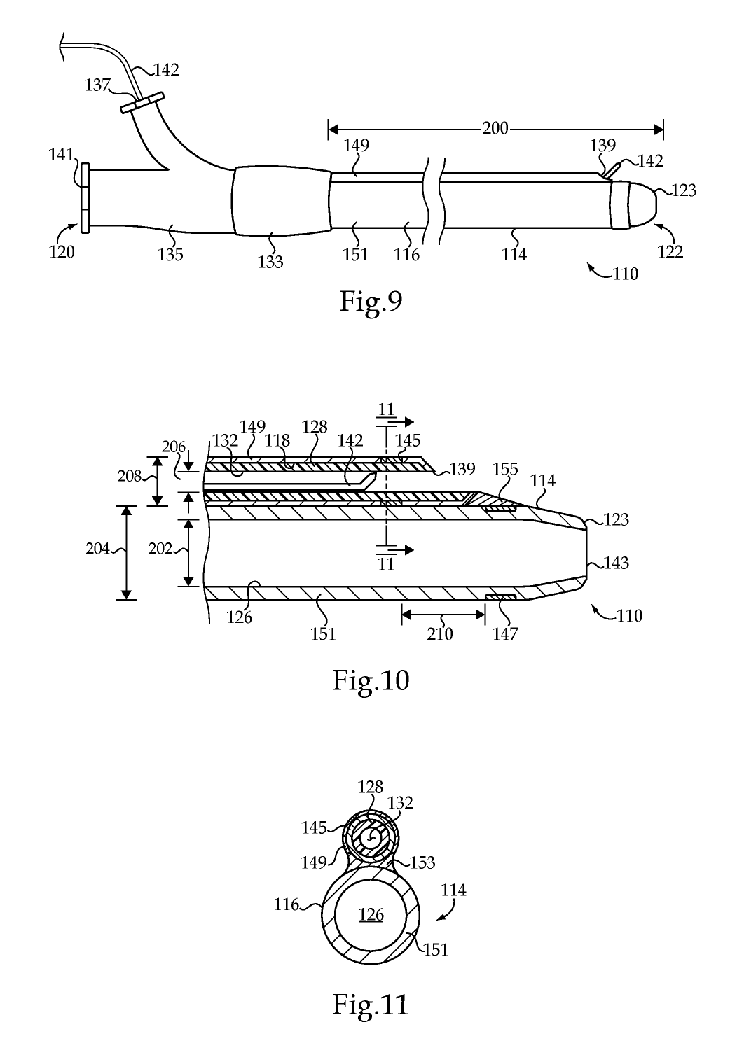

Referring now to FIG. 9, there is shown a lumen re-entry system 110 according to another embodiment and including a catheter 114 having an elongate tubular body 116 with a proximal end 120 and a distal end 122 that includes a distal tip 123. System 110 further includes a puncture wire 142 having a configuration and adapted for use in a manner the same or similar as that described in connection with previous embodiments. From the standpoint of materials, component geometry, and use it should be understood that system 110 is generally analogous to other embodiments contemplated herein, and various features of system 110 could be substituted for features of system 10, and vice versa, except where otherwise indicated or apparent from the present description.

Referring also now to FIG. 10, catheter 114 may further have formed therein a wire lumen 118, a working lumen 126, and a reduced wire lumen 132. Lumen 132 is defined by an abrasion resistant tubular liner 128 affixed to elongate tubular body 116, and having a fixed position therein. Reduced wire lumen 132 extends from an open proximal liner end 137 to an open distal liner end 139. Working lumen 126 extends from an open proximal end 141 to an open distal end 143. It may be noted that working lumen 126 is shown as extending in parallel with reduced wire lumen 132. In the illustrated embodiment, open distal liner end 139 has the general form of a side port, opening proximally of open distal end 143 of working lumen 126. In other embodiments, similar to that discussed above, each of reduced wire lumen 132 and working lumen 126 could open at distal tip 123. Catheter 114 further includes a manifold 133 forming fluid connections between open proximal end 137 and open distal end 139 and between open proximal end 141 and open distal end 143. Open distal ends 137 and 141 are located in a hub 135 in the illustrated embodiment.

In one practical implementation strategy elongate tubular body 114 is formed of a first polymer material, and liner 128 is formed of a different polymer material harder than the first polymer material. In alternative embodiments, liner 128 could be formed of a metallic material as discussed above. Liner 128 may further include one or more flexibility increasing cuts. Such cuts are not shown in FIGS. 9 and 10, however the implementation of one or more such cuts will be readily understood for system 110 in a manner generally analogous to that described in connection with foregoing embodiments.

Elongate tubular body 114 may further include a smaller diameter first shaft 149 defining wire lumen 118 and having abrasion resistant tubular liner 128 positioned therein. Body 116 further includes a larger diameter second shaft 151 attached to shaft 149 and defining working lumen 126. Catheter 114 may further include a first radiopaque marker 145 extending circumferentially around reduced wire lumen 132, and a second radiopaque marker extending circumferentially around working lumen 126. As can be seen from FIG. 10, first and second radiopaque markers 145 and 147 are offset from one another in a proximal to distal direction, with marker 145 being positioned proximal of marker 147. Markers 145 and 147 can also be understood to be offset from one another in a transverse direction. In a practical implementation strategy, marker 145 is both smaller in diameter and smaller in axial extent, in reference to a longitudinal axis of liner 128, than marker 147. Markers 145 and 147, due to their offset and enhanced by their different sizes, can enable a clinician to visualize an orientation of catheter 114 within a patent, for properly orienting puncture wire 142 for entering a subintimal space or exiting the same to re-enter a true lumen in a vessel in a patient. Those skilled in the art will thus readily appreciate how a clinician could apply a torque to proximal end 120, or near proximal end 120, of catheter 114 to rotate catheter 114 to position open distal end 139 in a desired orientation. In a practical implementation strategy, a spacing 210 between markers 145 and 147 may be from about 5 mm to about 15 mm. A working length 200 of catheter 114 extending from manifold 133 to distal tip 123 may be about 150 cm. An inner diameter dimension 202 of working lumen 126 may be about 0.04 inches or 1.02 mm, more particularly 0.037 inches or 0.94 mm in a practical implementation strategy. An outer diameter dimension 204 of second shaft 151 may be about 0.05 inches or 1.27 mm, more particularly 0.046 inches or 1.17 mm. An inner diameter dimension 206 of reduced wire lumen 132 may be about 0.02 inches or 0.51 mm, more particularly 0.017 inches or 0.43 mm. An outer diameter dimension 208 of first shaft 149 may be about 0.02 inches or 0.51 mm, more particularly 0.020 inches or 0.51 mm. As used herein, the term “about” should be understood in the context of conventional rounding to a consistent number of significant digits. Accordingly, “about 150” means from 145 to 154, and so on.

Referring also now to FIG. 11, there is shown a sectioned view taken along line 11-11 of FIG. 10. As noted above, shaft 149 may be attached to shaft 151. In a practical implementation strategy, shafts 149 and 151 are attached by melting material from which shafts 149 and 151 are formed, potentially with the inclusion of additional shaft material 153 therebetween. It may be noted from FIG. 11 that material of shaft 149 encases marker 145. It is contemplated that one practical strategy for making catheter 114 will include attaching marker 145 to liner 128, and attaching marker 147 to shaft 151, while each of liner 128 and shaft 151 is maintained as a separate piece. Markers 145 and 147 might be attached to the respective components via swagging, although the present disclosure is not thereby limited. With marker 145 attached to liner 128, liner 128 may be encased in material forming shaft 149. Liner 128 may be slid into shaft 149 as a preformed body, but material of shaft 149 could instead be applied via another strategy such as application in a molten state. With shaft 149, liner 128 and marker 145 assembled, they may be attached to shaft 151 such as via melting and permitting material of shaft 149 and/or 151 and additional material 153 to solidify, or via an adhesive. A distal end of liner 118 and the encasing material of shaft 149 may then be cut to establish open distal end 139. Additional, filler material 155 may be applied near open distal end 139 to provide a smooth transition distally of liner 128 toward distal tip 123, and potentially to encase marker 147. Open distal end 139 may have an inside shape with the form of a ramp to assist in deflection of the tip of wire 142 in a radially outward direction.

### INDUSTRIAL APPLICABILITY

The present disclosure is generally applicable to medical devices for use in percutaneous vascular procedures, or other procedures involving cavities, ducts, or canals of a patient. More specifically, the present disclosure is applicable to systems and methods for treating chronic total occlusion (CTO). Yet further, the present disclosure may be specifically applicable to systems and methods for entering the subintimal space of a vessel wall and re-entering the lumen defined by the vessel wall after the occlusion.

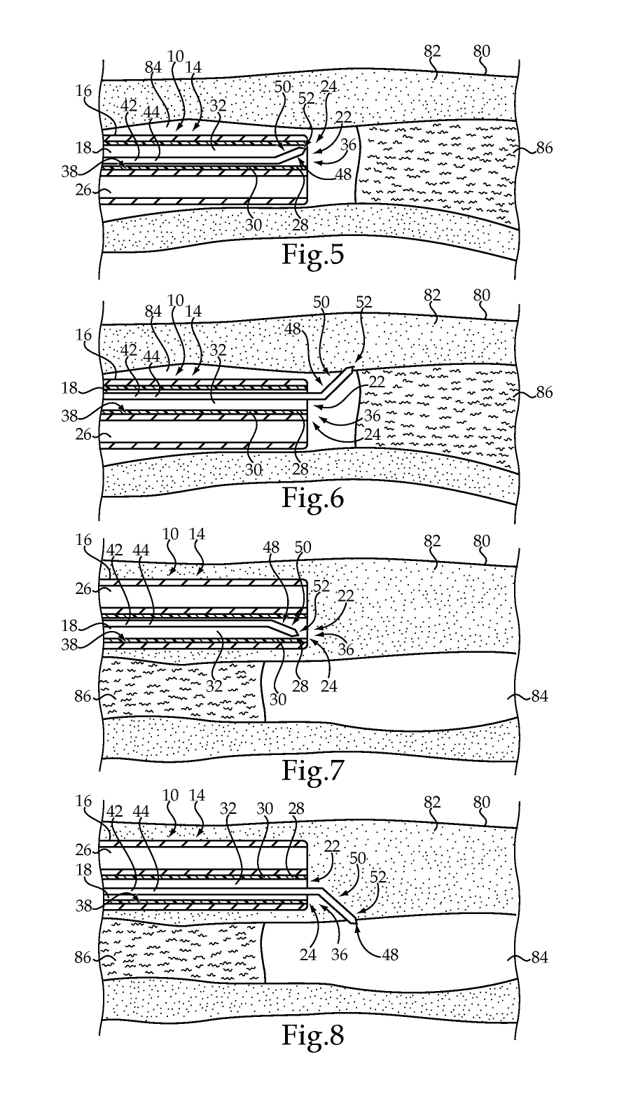

Referring to FIGS. 5-8, a percutaneous vascular procedure using the lumen re-entry system 10 of FIG. 1 will be described with reference to a vascular structure 80 of a patient. It will be appreciated that the present description is analogously applicable to the embodiment of FIGS. 9-11. The vascular structure 80, as should be appreciated, includes a vessel wall 82 defining a lumen 84. Although not shown, a clinician may position a needle, or introducer, through the skin of a patient to gain access to the vascular structure 80. At a first stage of the procedure, a clinician may insert a conventional wire guide through a tube of the introducer and into the vascular structure 80. While attempting to cross an occlusion 86, the clinician may inadvertently, or intentionally, penetrate into the vessel wall 82 or, more specifically, the subintimal space of the vessel wall 82.

For example, the lumen re-entry system 10 may be used for intentionally entering the vessel wall 82 from the lumen 84. For example, the catheter 14 may be advanced through the lumen 84 of the patient vessel 80 in a conventional manner. If a conventional wire guide is unsuccessful in crossing the occlusion 86, the conventional wire guide may be removed and the puncture wire 42 may be axially, and telescopically, advanced through the reduced wire lumen 32 and, as shown in FIG. 5, the puncture tip 52 of the puncture wire 42 may be oriented such that the puncture tip 52 faces the wall 82. While properly oriented, the puncture wire 42 may be axially advanced further through the reduced wire lumen 32 such that the angled distal segment 50 is advanced through the distal opening 22 and the puncture tip 52 penetrates the wall 82, as shown in FIG. 6. The catheter 14 may then enter the wall 82 over the puncture wire 42, and/or the puncture wire 42 may be exchanged for a conventional wire guide or other medical device.

As shown, the puncture wire 42 is axially advanced through the catheter 14 with the angled distal segment 50 oriented at an angle between about 10 degrees to about 90 degrees relative to the central longitudinal axis A₁ of the puncture wire 42. Thus, during the axial advancement, the puncture tip 52 of the puncture wire 42 may contact the abrasion resistant tubular liner 28. As stated above, the abrasion resistant tubular liner 28 has a durometer higher than the elongate tubular body 16 of the catheter 14 and, thus, is more resistant to abrasion and/or scoring due to contact by the puncture tip 52.

As shown in FIG. 7, the lumen re-entry system 10 may also be used to re-enter the true lumen 84 beyond the occlusion 86. Specifically, the catheter 14 may be advanced through the wall 82 of the patient vessel 80. The puncture wire 42 may then be axially advanced through the reduced wire lumen 32 with the angled distal segment 50 oriented at an angle between about 10 degrees to about 90 degrees relative to the central longitudinal axis A₁ of the puncture wire 42. As stated above, the puncture tip 52 of the puncture wire 42 may contact the abrasion resistant tubular liner 28 during the advancement of the puncture wire 42 through the catheter 14. With the puncture tip 52 of the puncture wire 42 oriented such that the puncture tip 52 faces the lumen 84 of the patient vessel 80, the true lumen 84 may be re-entered with the puncture wire 42 by axially advancing the puncture wire 42 further through the reduced wire lumen 32 such that the angled distal segment 50 is advanced through the distal opening 22 and the puncture tip 52 penetrates the wall 82. The catheter 14 may then be advanced through the puncture made by the puncture tip 52 of the puncture wire 42 and into the true lumen 84.

It should be appreciated that a lubricious polymer coating on one or more of the catheter 14, abrasion resistant tubular liner 28, and puncture wire 42 may assist in reducing friction as the components are moved within the patient vasculature 80 and relative to one another. Further, one or more flexibility increasing cuts, such as cuts 62 described above with respect to abrasion resistant tubular liner 60 of FIGS. 3 and 4, may improve flexing of the abrasion resistant tubular liner 28 and, thus, catheter 14, during catheter movement. It should also be appreciated that radiopaque markers and/or additional components and devices that facilitate imaging assisted advancement may be incorporated into the lumen re-entry system 10 described herein. Such imaging assisted advancement may be particularly useful in properly orienting the angled distal segment 50, and determining where to exit and re-enter the true lumen 84 relative to the occlusion 86.

The lumen re-entry system 10 described herein provides a means for effectively entering the subintimal space of a patient vessel 80 and/or re-entering a patient lumen 84 after a wire guide has inadvertently, or intentionally, advanced into the subintimal space, such as while attempting to cross an occlusion 86. In particular, a puncture wire 42 having a stiff, angled distal segment 50 may be used by a clinician in conjunction with the catheter 14 described herein to quickly and efficiently perform the percutaneous procedures. The abrasion resistant tubular liner 28 effectively reduces contact between the puncture tip 52 and the wall 38 defining the wire lumen 18 and, as such, reduces the risk of damage and/or failure of the lumen re-entry system 10. In particular, the abrasion resistant tubular liner 28 minimizes the risk of puncturing and/or tearing the catheter wall 38. Although the components are described with respect to a lumen re-entry procedure, it should be appreciated that the components may be broadly applicable to a wide variety of percutaneous vascular procedures beyond the scope of CTO treatment.

It should be understood that the above description is intended for illustrative purposes only, and is not intended to limit the scope of the present disclosure in any way. Thus, those skilled in the art will appreciate that other aspects of the disclosure can be obtained from a study of the drawings, the disclosure and the appended claims.

1.-30. (canceled)

31. A vessel lumen re-entry system configured to create a new lumen through a subintimal space that bypasses a chronic total occlusion in the vessel lumen, comprising:
a dual lumen catheter defining a working lumen extending in parallel with a puncture wire lumen;
a metallic tubular liner affixed within the puncture wire lumen, and defining at least one flexibility increasing cut entirely through a wall of the metallic tubular liner;
a puncture wire at least partially positioned in the metallic tubular liner and having an angled distal segment terminating in a puncture tip with a sharp point, and the puncture tip contacts the metallic tubular liner when the puncture tip is positioned within the puncture wire lumen, and wherein the puncture tip is oriented at an angle between about 10 degrees to about 90 degrees relative to a central longitudinal axis of the puncture wire; and
first and second radiopaque markers attached to the dual lumen catheter and being separated by a longitudinal offset distance and by a transverse offset distance so as to enable a clinician to visualize an orientation of the dual lumen catheter within a patient.

32. The vessel lumen re-entry system of claim 31 wherein the first radiopaque marker extends circumferentially around the puncture wire lumen; and
the second radiopaque marker extends circumferentially around the working lumen.

33. The vessel lumen re-entry system of claim 32 wherein the first radiopaque marker has a smaller diameter than the second radiopaque marker.

34. The vessel lumen re-entry system of claim 31 wherein the working lumen has a larger diameter than the puncture wire lumen.

35. The vessel lumen re-entry system of claim 34 wherein the first radiopaque marker extends circumferentially around the puncture wire lumen; and
the second radiopaque marker extends circumferentially around the working lumen.

36. The vessel lumen re-entry system of claim 31 wherein an open distal end of the puncture wire lumen opens proximally of an open distal end of the working lumen.

37. The vessel lumen re-entry system of claim 31 wherein the puncture wire lumen is defined by a first shaft;
the working lumen is defined by a second shaft; and
the first shaft is attached to the second shaft by an additional material.

38. The vessel lumen re-entry system of claim 37 wherein the working lumen has a larger diameter than the puncture wire lumen.

39. The vessel lumen re-entry system of claim 31 wherein the at least one flexibility increasing cut includes a single continuous spiral cut.

40. The vessel lumen re-entry system of claim 31 wherein the first radiopaque marker extends circumferentially around the puncture wire lumen;
the second radiopaque marker extends circumferentially around the working lumen;
the first radiopaque marker has a smaller diameter than the second radiopaque marker;
the working lumen has a larger diameter than the puncture wire lumen; and
an open distal end of the puncture wire lumen opens proximally of an open distal end of the working lumen.

41. A method of creating a new lumen through a subintimal space bypassing a chronic total occlusion in a vessel lumen with a vessel lumen re-entry system that includes a dual lumen catheter defining a working lumen extending in parallel with a puncture wire lumen; a metallic tubular liner affixed within the puncture wire lumen, and defining at least one flexibility increasing cut entirely through a wall of the metallic tubular liner; a puncture wire at least partially positioned in the metallic tubular liner and having an angled distal segment terminating in a puncture tip with a sharp point, and the puncture tip contacts the metallic tubular liner when the puncture tip is positioned within the puncture wire lumen, and wherein the puncture tip is oriented at an angle between about 10 degrees to about 90 degrees relative to a central longitudinal axis of the puncture wire; and first and second radiopaque markers attached to the dual lumen catheter and being separated by a longitudinal offset distance and by a transverse offset distance so as to enable a clinician to visualize an orientation of the dual lumen catheter within a patient, the method comprising the steps of:
moving the puncture tip of the puncture wire from within the vessel lumen on a first side of the chronic total occlusion, through a vessel wall and into the subintimal space;
advancing the dual lumen catheter over the puncture wire and into the subintimal space;
orienting the puncture tip in the subintimal space to face the vessel lumen;
moving the puncture tip from the subintimal space, through the vessel wall and back into the vessel lumen on an opposite side of the chronic total occlusion; and
advancing the dual lumen catheter over the puncture wire back into the vessel lumen on the opposite side of the chronic total occlusion.

42. The method of claim 41 wherein the orienting step includes identifying a relative positioning of the first and second radiopaque markers.

43. The method of claim 42 wherein the orientating step includes applying a torque to the dual lumen catheter.

44. The method of claim 41 including protecting the puncture wire lumen against damage by movement of the puncture wire therein with the metallic tubular liner.

45. The method of claim 41 including exchanging the puncture wire for a wire guide after the dual lumen catheter is advanced into the subintimal space.
